(19) United States
(12) Patent Application Publication
(10) Pub. No.: US 2025/0057767 A1
(43) Pub. Date: Feb. 20, 2025

(54) COMPOSITION COMPRISING HYBRID EXOSOME

(71) Applicants: COSMECCA KOREA CO., LTD., Eumseong gun (KR); GFC Life Science Co., Ltd., Hwaseong-si (KR); KOREA BASIC SCIENCE INSTITUTE, Daejeon (KR)

(72) Inventors: Ji Soo Ryu, Anyang-si (KR); A Reum Jang, Seoul (KR); Jae Yong Seo, Cheongju-si (KR); Hyun Dae Cho, Cheongju-si (KR); Hee Cheol Kang, Hwaseong-si (KR); Mi Jung Kim, Seoul (KR); En Young Jeong, Hwaseong-si (KR); Hye Sun Park, Sejong-si (KR); Kwan Soo Hong, Cheongju-si (KR); Hyunseung Lee, Daejeon (KR)

(21) Appl. No.: 18/807,111

(22) Filed: Aug. 16, 2024

(30) Foreign Application Priority Data
Aug. 18, 2023 (KR) 10-2023-0108099

(51) Int. Cl. A61K 9/127; A61K 8/14; A61K 8/9789; A61K 9/50

(52) U.S. Cl. CPC A61K 9/1271; A61K 8/14; A61K 8/9789; A61K 9/1277; A61K 9/5068

(57) ABSTRACT

The present invention relates to hybrid exosomes, a use of the same, and a preparation method of the same, and the hybrid exosomes provided from the present invention have excellent skin inflammation amelioration, skin wrinkle amelioration, skin moisturization efficacy, and skin regeneration efficacy compared to using hydrangea-derived exosomes and liposomes alone, and thus can be effectively utilized as a cosmetic composition containing the same as an active ingredient. In addition, the hybrid exosomes have better stability and biocompatibility compared to existing exosomes.

### CROSS-REFERENCE TO RELATED APPLICATION

This application claims priority to and the benefit of Korean Patent Application No. 2023-0108099, filed on Aug. 18, 2023, the disclosure of which is incorporated herein by reference in its entirety.

### BACKGROUND

### 1. Field of the Invention

The present invention relates to a composition including hybrid exosomes.

### 2. Discussion of Related Art

The skin, which is the outermost layer of the human body, is one of the tissues that are most easily affected by ultraviolet rays, and as outdoor activities increase, the exposure time to ultraviolet rays is prolonged, and thus the number of patients suffering from skin aging and skin diseases is continuously increasing. Ultraviolet rays are light rays with a wavelength of 200 to 400 nm that cause skin damage and aging, and it is known that ultraviolet rays cause most cosmetic and medical problems that occur in the skin. When exposed to ultraviolet rays, excess nitric oxide (NO) is produced in the skin, and the produced NO damages the lipids, proteins, nucleic acids, enzymes, and the like that make up the skin, thereby promoting the production of inflammation mediators. When this inflammatory response continues, the skin is damaged. Accordingly, many studies are being conducted to develop substances that can maximize the skin improvement effects through changes in the activity of anti-inflammation, skin moisturization, and collagen synthesis-related factors. In addition, research on and consumer demand for cosmetic compositions that can be used by not only maximizing the above advantages but also minimizing harmful factors such as skin irritation and cytotoxicity are also increasing.

Exosomes are substances that are secreted by all living cells, including animal, plant, and microbial cells, and are representative extracellular vesicles with a size of 50 to 200 nm, composed of a phospholipid bilayer membrane. Exosomes may be delivered to target cells, and they simultaneously transport various secondary metabolites, membrane proteins, and genetic information materials (DNA, RNA, proteins) that cells have, thereby facilitating signaling in cells. In addition, since exosomes include components similar to those of cell membranes, they are highly biocompatible, easily penetrate cells, and exhibit a high absorption rate. Currently, active research is being conducted on exosomes as a delivery vehicle in the pharmaceutical and biological fields, and recently, the skin care and cosmetics industries have also been paying attention to these properties of exosomes and are showing increasing interest in exosomes. Among these exosomes, plant-derived exosomes have been reported to have less cytotoxicity than animal-derived exosomes, and have an advantage in terms of raw material supply. In particular, the cosmetics industry is paying attention to plant-derived exosomes due to the demand of consumers who are highly concerned about the environment and prefer vegan products. However, unlike other vesicles, these exosomes have limitations in capturing effective substances, and research is required to improve this.

Recently, research on new biomaterials developed by biologically or chemically modifying exosomes has been actively conducted. In particular, hybrid exosomes have been developed by changing the properties of exosomes through direct membrane fusion of exosomes and synthetic liposomes and improving the shortcomings of exosomes and synthetic liposomes. The development a skin delivery system by hybridizing exosomes derived from Centella asiatica with liposomes, where hybrid lipids were developed by a high-pressure emulsification method to form stable vesicle membranes without affecting the inherent properties of nanoparticles has been disclosed. However, the hybridization of exosomes obtained from hydrangea with liposomes encapsulating effective substances at an optimal ratio to exhibit excellent efficacy has not been disclosed.

### RELATED ART DOCUMENTS

### Patent Documents

(Patent Document 0001) Korean Patent Publication 10-2020-0145115
(Patent Document 0002) Korean Registered Patent Publication 10-2450893

### SUMMARY OF THE INVENTION

The present inventors conducted research to develop a cosmetic composition with improved anti-inflammatory, anti-aging, and moisturizing effects, and confirmed that hybrid exosomes obtained by hybridizing hydrangea-derived exosomes and liposomes have excellent effects in skin inflammation amelioration, skin elasticity improvement, skin wrinkle amelioration, skin regeneration, and skin moisturization, thereby completing the present invention.

An object of the present invention is to provide a cosmetic composition for improving skin conditions, including hybrid exosomes as an active ingredient.

In addition, another object of the present invention is to provide a composition for delivering drugs or physiologically active substances encapsulated in the hybrid exosomes.

In addition, still another object of the present invention is to provide a method of producing hybrid exosomes.

The present inventors confirmed that the hybrid exosomes are effective in improving skin conditions. Therefore, the present invention provides hybrid exosomes in which hydrangea-derived exosomes and liposomes are hybridized.

In the present invention, “exosomes” refer to nano-sized vesicles having a membrane structure secreted or released from hydrangea into the extracellular space, and are also defined as exosome-like vesicles or exosome-like particles.

In the present invention, “hydrangea-derived exosomes” encompass all exosomes isolated from, for example, a hydrangea culture solution or a hydrangea fermentation product, or an equivalent hydrangea biological solution, or secreted and/or released from hydrangea itself. Various types of hydrangea that are used or may be used in the art in the future may be used to produce hydrangea-derived exosomes, and it should be understood that the types of hydrangea used in the examples are examples of hydrangea that may be used in the present invention, and it is clearly stated that the present invention is not limited thereto.

In the present invention, “liposomes” refer to microvesicles having a hydrophilic space inside and a closed double lipid membrane outside, where the liposome has a structure like a hollow droplet with a double layer of phosphorylated lipids generated when phospholipids are added to an aqueous solution.

In the present invention, “hybrid exosomes” refer to exosomes obtained by hybridizing the hydrangea-derived exosomes and liposomes.

In one embodiment of the present invention, the weight ratio of the exosome and the liposome may be 16:1 to 1:2, 8:1 to 1:2, 4:1 to 1:2, 2:1 to 1:2, for example, 1:1 to 1:2.

In one embodiment of the present invention, the diameter of the hybrid exosomes may be 50 to 400 nm, 100 to 300 nm, for example, 100 to 200 nm.

In the present invention, “skin condition improvement” refers to reducing the degree of symptoms related to worsening skin conditions, and the skin conditions may be parameters related to skin inflammation, skin elasticity, skin wrinkles, skin regeneration, and skin moisturization.

In the present invention, “anti-inflammation” or “skin inflammation amelioration” refers to suppressing or ameliorating inflammation, and the inflammation is one of the defense responses of living tissues to a certain stimulus, and refers to a complex lesion that causes three things: tissue degeneration, circulatory disorders and exudation, and tissue proliferation. More specifically, inflammation is a part of innate immunity, and like other animals, human innate immunity recognizes cell surface patterns that are specifically present in pathogens. Phagocytes recognize cells with such surfaces as non-self and attack pathogens. When pathogens break through the physical barrier of the body and enter, an inflammatory response occurs. An inflammatory response is a non-specific defense action that creates a hostile environment for microorganisms that have invaded an wound site. In an inflammatory response, when a wound occurs or an external infectious agent enters the body, white blood cells responsible for the initial immune response rush in and express cytokines. Therefore, the cytokine expression level in cells serves as an indicator of the activation of an inflammatory response.

Specifically, “skin elasticity improvement” refers to maintaining or increasing the elasticity of the skin, in addition to the effects of contracting sagging skin tissues by increasing the volume of skin adipose tissues, such as by promoting collagen synthesis.

In addition, “skin wrinkles” refer fine lines that are generated due to aging of the skin, and may be caused by genetic causes, a decrease in collagen and elastin in the skin's dermis, external environments, and the like. Therefore, the term “skin wrinkle improvement” used herein refers to suppressing or inhibiting the generation of wrinkles on the skin, or alleviating wrinkles that have already been generated.

In addition, “skin regeneration” refers to the recovery of skin tissue damaged by external and internal causes. The external causes include ultraviolet rays, external pollutants, wounds, trauma, and the like, and the internal causes include diseases, stress, and the like.

In the present invention, “skin moisturization” refers to inhibiting or suppressing the decrease in moisture of the skin or increasing the moisture content of the skin to make the skin surface smooth and provide gloss.

In one embodiment of the present invention, the ability to inhibit the production of nitric oxide (NO), which is an inflammation-related factor, and the ability to inhibit the production of tumor necrosis factor (TNF)-α were evaluated, and it was confirmed that the effect of inhibiting the production of NO and TNF-α of the hybrid exosomes was better compared to the case where the hydrangea-derived exosomes were used alone.

In addition, in one embodiment of the present invention, as a result of evaluating the ability to inhibit the production of matrix metalloproteinase (MMP)-1, a skin elasticity-related protein, it was confirmed that the wrinkle improvement effect of the hybrid exosomes was better, and as a result of evaluating the ability to produce hyaluronic acid, a moisturizing factor, it was confirmed that the moisturizing effect of the hybrid exosomes was better.

In one embodiment of the present invention, as a result of confirming the expression of proteinase-activated receptor (PAR)-2, thymic stromal lymphopoietin (TSLP), and interleukin (IL)-6, which are factors involved in the inflammatory response to ultraviolet rays, it was confirmed that the expression of all of PAR-2, TSLP, and IL-6 was suppressed when hybrid exosomes were used.

Therefore, hybrid exosomes can exhibit one or more effects selected from the group consisting of skin inflammation amelioration, skin elasticity improvement, skin wrinkle amelioration, skin regeneration, and skin moisturization effects.

In addition, the present invention provides a composition for improving skin conditions, including hybrid exosomes as an active ingredient.

In one embodiment of the present invention, the hybrid exosomes may be included in an amount of 0.01% to 20% by weight, 0.1% to 15% by weight, for example, 0.1% to 10% by weight, based on the total weight of the composition. When the content of the hybrid exosomes is less than 0.01% by weight based on the total weight of the composition, efficacy may be minimal, and even when the hybrid exosomes are included in an amount of more than 20% by weight, it is difficult to expect an increase in the effects of the hybrid exosomes in proportion to the added amount, which is economically undesirable, and so there is a problem of significantly increasing the unit price.

In one embodiment of the present invention, the liposomes may include terpineol. In the present invention, the terpineol may improve the permeability of polar and water-soluble substances, thereby increasing the fluidity of the membrane. For example, the terpineol may be at least one selected from the group consisting of α-terpineol, β-terpineol, γ-terpineol, δ-terpincol, and terpinen-4-ol. Preferably, the terpineol may be terpinen-4-ol, but is not limited thereto.

In the present invention, the terpinen-4-ol is an isomer of terpineol, and is a main ingredient accounting for 40% to 60% of tea tree oil, and is a component extracted from leaves, branches, and bark, separated, and purified. The terpinen-4-ol has excellent antibacterial and anti-inflammatory effects, and may be used as a substance that improves the permeability of polar and water-soluble drugs.

In one embodiment of the present invention, the terpineol may be included in an amount of 0.01% to 10% by weight, 0.05% to 5% by weight, 0.1% to 1% by weight, for example, 0.05% to 1% by weight, based on the total weight of the exosomes. When terpineol is included in an amount of less than 0.01% by weight, the effect may be minimal, and when it is included in an amount of more than 10% by weight, there may be a problem in forming hybrid exosomes.

In addition, in one embodiment of the present invention, the liposomes may further include one or more selected from the group consisting of ethanolamide, lecithin, a surfactant, and a ceramide.

In the present invention, the ethanolamide may be secondary carboxamides composed of amides of alkyl carboxylic acids and ethanolamine and may be one or more selected from the group consisting of acetamide monoethanolamine (MEA), azelamide MEA, babassuamide MEA, behenamide MEA, C16-22 acid amide MEA, cocamide MEA, cocamide methyl MEA, cocamidopropyl betainamide MEA chloride, hydroxypropyl bispalmitamide MEA, hydroxystearamide MEA, isostearamide MEA, lactamide MEA, lauramide MEA, linoleamide MEA, myristamide MEA, oatamide MEA, oleamide MEA, oliveamide MEA, palm kernelamide MEA, palmamide MEA, palmitamide MEA, pantothenamide MEA, peanutamide MEA, ricinoleamide MEA, stearamide MEA, tallowamide MEA, and undecylenamide MEA. Preferably, the ethanolamide may be azclamide MEA, but is not limited thereto.

In the present invention, the ethanolamide may be included in an amount of 0.001% to 5% by weight, 0.005% to 5% by weight, 0.01% to 1% by weight, for example, 0.01% to 0.5% by weight, based on the total weight of the exosomes. When ethanolamide is included in an amount of less than 0.001% by weight, the effect may be minimal, and when it is included in an amount of more than 5% by weight, there may be a problem in forming hybrid exosomes.

In the present invention, as the lecithin, a conventional lecithin may be used. For example, commercially available soybean-extracted lecithin, which has a fatty acid chain of 12 to 24 carbon atoms and is composed of a mixture of phospholipids such as phosphatidylcholine, phosphatidylethanolamine, phosphatidylserine, phosphatidylglycerol, and phosphatidylinositol and other fatty acids, may be used, but the lecithin is not limited thereto. For example, the lecithin in the present invention may be hydrogenated lecithin.

In the present invention, the lecithin may be included in an amount of 0.1% to 20% by weight, 0.1% to 10% by weight, for example, 0.1% to 5% by weight, based on the total weight of the exosomes. In the above range, the exosomes may be stable without deteriorating the physical properties of the phospholipid layer. When lecithin is included in an amount less than 0.1% by weight or more than 20% by weight, there may be a problem in forming hybrid exosomes.

In the present invention, the surfactant may include one or more selected from dipotassium glycyrrhizate, disodium glycyrrhizate, and ammonium glycyrrhizate and one or more selected from substances having a fatty acid chain of 6 to 20 carbon atoms and forming an ester bond with polyglyceryl. The surfactant may be included in an amount of 0.01% to 20% by weight, 0.05% to 10% by weight, for example, 0.1% to 5% by weight, based on the total weight of the exosomes. When the surfactant is included in an amount of more than 20% by weight, there may be a problem that efficacy may be insufficient due to a decrease in the amount of components incorporated into the hybrid exosomes due to an excess of membrane components.

In the present invention, the ceramide may serve as a stabilizing agent that stabilizes the phospholipid layer and as a skin component that strengthens the skin barrier. For example, the ceramide may be included in an amount of 0.01% to 10% by weight, 0.01% to 5% by weight, or 0.01% to 1% by weight based on the total weight of the exosomes, and stable liposomes may be obtained within the above range. When the ceramide is included in an amount of more than 10% by weight, it may precipitate, not only affecting stability but also causing problems in the formation of hybrid exosomes.

Meanwhile, the composition for improving skin conditions in the present invention may be used in various forms such as a cosmetic composition, a food composition, a pharmaceutical composition, and the like. For example, in one embodiment of the present invention, the composition may be a cosmetic composition.

The cosmetic composition for improving skin conditions of the present invention may be made into at least one formulation selected from the group consisting of lotion, liquid, cream, soaked masks, gel, aerosol, and powder.

The lotion refers to a formulation made into a viscous liquid state by mixing an active ingredient of a cosmetic composition with an emulsifier and the like and homogenizing oily and aqueous ingredients, and the cream refers to a formulation made into a semi-solid state by mixing an active ingredient of a cosmetic composition with an emulsifier and the like and homogenizing oily and aqueous ingredients. In addition, the liquid is a formulation made into a liquid state by dissolving an active ingredient of a cosmetic composition and other ingredients in a solvent or the like, and a gel refers to a semi-solid state made of organic molecules with a large molecular weight that has been permeated with a liquid.

The soaked mask is a formulation made by soaking a support such as non-woven fabric with lotions, creams, liquids, gels, and the like, and may be made into patches, mask packs, mask sheets, and the like. The aerosol refers to a formulation in which an original liquid is designed to be sprayed in the form of mist, foam, or the like using the pressure of a propellant (liquefied gas, compressed gas, etc.) filled in the same container or a different container. In addition, the powder refers to a formulation made into a homogeneous powder or fine particle form, and excipients or the like may be used.

For example, the cosmetic composition for improving skin conditions of the present invention may be made into a lotion or a cream, or may be applied to or soaked into at least one surface of a patch, a mask pack, or a mask sheet.

Meanwhile, the cosmetic composition of the present invention is used for the purpose of skin inflammation amelioration, skin elasticity improvement, skin wrinkle amelioration, skin regeneration, and/or skin moisturization, and may be prepared in any form commonly prepared in the art. For example, it may be prepared as a patch, a mask pack, a mask sheet, an emollient toner, a nourishing toner, an astringent toner, a nourishing cream, a massage cream, an eye cream, a cleansing cream, an essence, an eye essence, a cleansing lotion, a cleansing foam, a cleansing water, a sunscreen, a lipstick, a soap, a shampoo, a surfactant-containing cleanser, a bath agent, a body lotion, a body cream, a body oil, a body essence, a body cleanser, a hair dye, a hair tonic, and the like, but is not limited thereto.

Meanwhile, the cosmetic composition for improving skin conditions of one embodiment of the present invention may further include ingredients commonly used in cosmetic compositions, such as moisturizers, antioxidants, oily ingredients, ultraviolet absorbers, emulsifiers, surfactants, thickeners, alcohols, powdery ingredients, colorants, aqueous ingredients, water, various skin nutrients, and the like, as needed, within a range that does not impair the effects of the present invention.

In addition, the cosmetic composition of one embodiment of the present invention may be used in combination with skin improving agents, antioxidants and/or moisturizers that have been used in the past, in addition to the hydrangea-derived exosomes, as long as the effects (skin inflammation amelioration, skin elasticity improvement, skin wrinkle amelioration, skin regeneration, skin moisturization, etc.) are not impaired. For example, the hydrangea-derived exosomes of the present invention may be supported on or mixed with at least one of a hydrogel, hyaluronic acid, a hyaluronic acid salt (e.g., sodium hyaluronate), or a hyaluronic acid gel. In the cosmetic composition of one embodiment of the present invention, the type of the hydrogel is not limited, but preferably, it may be a hydrogel obtained by dispersing a gelling polymer in a polyhydric alcohol. The gelling polymer may be at least one selected from the group consisting of pluronics, purified agar, agarose, gellan gum, alginic acid, carrageenan, cassia gum, xanthan gum, galactomannan, glucomannan, pectin, cellulose, guar gum, and locust bean gum, and the polyhydric alcohol may be at least one selected from the group consisting of ethylene glycol, propylene glycol, 1,3-butylene glycol, isobutylene glycol, dipropylene glycol, sorbitol, xylitol, and glycerin.

In addition, the cosmetic composition of the present invention may further include one or more cosmetically acceptable carriers that are blended with general skin cosmetics, and may be appropriately blended with conventional ingredients, for example, oils, water, surfactants, moisturizers, lower alcohols, thickeners, chelating agents, pigments, preservatives, fragrances, and the like, but is not limited thereto.

When the formulation of the present invention is powder or an aerosol, lactose, talc, silica, aluminum hydroxide, calcium silicate, polyamide powder or a mixture thereof may be used as a carrier ingredient, and particularly, an aerosol may further include a propellant such as chlorofluorohydrocarbon, propane/butane or dimethyl ether.

When the formulation of the present invention is a cream or gel, animal fiber, plant fiber, wax, paraffin, starch, tragacanth, cellulose derivatives, polyethylene glycol, silicone, bentonite, silica, talc, or zinc oxide may be used as a carrier ingredient.

When the formulation of the present invention is a solution or emulsion among liquids, a solvent, a solvating agent, or an emulsifying agent may be used as a carrier ingredient, and for example, water, ethanol, isopropanol, ethyl carbonate, ethyl acetate, benzyl alcohol, benzyl benzoate, propylene glycol, 1,3-butyl glycol oil, glycerol aliphatic ester, polyethylene glycol, or fatty acid ester of sorbitan may be used.

When the formulation of the present invention is a suspension among liquids, liquid diluents such as water, ethanol, or propylene glycol, suspending agents such as ethoxylated isostearyl alcohol, polyoxyethylene sorbitol ester, and polyoxyethylene sorbitan ester, microcrystalline cellulose, aluminum metahydroxide, bentonite, agarose, or tragacanth, or the like may be used as a carrier ingredient.

In addition, the present invention provides a composition for delivery of a drug or a bioactive substance, including hybrid exosomes; and a drug or a bioactive substance encapsulated within the hybrid exosomes.

In the composition for delivery, all of the contents described above about the hybrid exosomes and the composition including the same may be applied as is.

In the present invention, “drug or bioactive substance” refers to any drug or bioactive substance that may be encapsulated in the hybrid exosomes of the present invention and delivered to a target site. For example, the drug or bioactive substance may be a peptide, a protein, an anticancer agent, an anti-inflammatory analgesic, an antibiotic, an antimicrobial agent, a hormone, a gene, or a vaccine, but is not limited thereto.

In the present invention, the hybrid exosomes are newly formed vesicles formed by physical membrane fusion of hydrangea exosomes and liposomes encapsulating an active substance, and not only change the membrane of the exosomes, but also encapsulate functional biomolecules inside the synthesized liposomes into the exosomes, thereby improving stability.

In addition, the hybrid exosomes of the present invention are characterized in that they compensate for the disadvantages of existing hydrangea-derived exosomes, which have excellent biocompatibility due to low immunogenicity because they contain various substances derived from living organisms, but have limited encapsulation of effective substances, and improve the disadvantages of liposomes, which are artificial synthetic substances with relatively low biocompatibility compared to exosomes. Therefore, the hybrid exosomes of the present invention are stable and have excellent biocompatibility compared to using hydrangea-derived exosomes and liposomes alone.

In one embodiment of the present invention, the hybrid exosomes may include terpincol. For example, the hybrid exosomes may have a form in which hydrangea-derived exosomes and liposomes encapsulating terpinen-4-ol and azelamide MEA are combined, but are not limited thereto. In the hybrid exosomes, terpinen-4-ol may be included in the lipid bilayer, and azelamide MEA may be included in the aqueous phase.

The hybrid exosomes contain terpineol, which is a substance that improves the permeability of polar and water-soluble drugs, thereby increasing the fluidity of the membrane, making it easier to pass through the stratum corneum compared to existing liposomes, and effectively allowing active ingredients to penetrate into the skin.

The present invention provides a method of preparing hybrid exosomes, including fusing hydrangea-derived exosomes and liposomes.

In the preparation method, all of the contents described above about the hybrid exosomes and the composition including the same may be applied as is.

In one embodiment of the present invention, the hydrangea-derived exosomes may be prepared by the following method: (a) adding distilled water to hydrangea leaf raw material at a weight ratio of 20:1, grinding the material, and stabilizing the ground material at 4° C. for 24 hours; (b) centrifuging the ground material using a centrifuge, and then taking only the supernatant of the ground solution to separate and remove dead cells and foreign substances; (c) centrifuging the supernatant of the ground solution using an ultracentrifuge, and then taking only the supernatant to separate and remove dead cells and residues; (d) filtering the supernatant to remove residues; (c) centrifuging the supernatant using an ultracentrifuge, and then settling exosomes to form a first pellet layer; (f) re-centrifuging the supernatant using an ultracentrifuge, and then settling additional exosomes to form a second pellet layer; and (g) resuspending the first and second pellet layers in purified water to separate exosomes from hydrangea leaves.

In addition, in one embodiment of the present invention, the liposomes may be prepared by the following method: (a) dissolving a phospholipid layer including ethanolamide, lecithin, a surfactant, and a ceramide; (b) dispersing terpineol in the phospholipid layer; (c) dispersing the phospholipid layer in purified water; and (d) homogenizing liposomes into a nano-size using a high-pressure disperser.

The above step (b) is a step for stabilizing an oil phase by dispersing terpineol in the phospholipid layer, and may be performed using a mixer, and typically, terpincol may be dispersed by stirring at 1,000 to 3,000 rpm for five minutes at 80° C. or higher.

The above step (c) is a step for dispersing the phospholipid layer in purified water to perform primary emulsification, and may be performed using a mixer, and typically, the phospholipid layer may be emulsified by stirring at 1,000 to 3,000 rpm for 10 minutes at 80° C. or higher.

The above step (d) is a step for preparing liposomes through secondary emulsification, and may be performed using a high-pressure disperser. The high-pressure disperser is used for the purpose of obtaining uniform nano-sized liposomes, and emulsification may be performed using high pressure. The secondary emulsification may be performed at room temperature using a high-pressure disperser at a pressure of 500 to 1,500 bar.

In one embodiment of the present invention, the weight ratio of the exosomes and liposomes may be 16:1 to 1:2, 8:1 to 1:2, 4:1 to 1:2, 2:1 to 1:2, for example, 1:1 to 1:2.

The advantages and features of the present invention and the method for achieving them will become clear with reference to the embodiments described in detail below. However, the present invention is not limited to the embodiments disclosed below, but may be implemented in various different forms, and these embodiments are provided only to make the disclosure of the present invention complete and to fully inform a person having ordinary skill in the art to which the present invention belongs of the scope of the invention, and the present invention is defined only by the scope of the claims.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 shows a schematic diagram illustrating a method of preparing hybrid exosomes according to the present invention;

FIG. 2 shows a graph illustrating changes according to liposome content according to the present invention;

FIG. 3A shows a graph illustrating changes according to exosome content according to the present invention;

FIG. 3B shows a graph illustrating the fluorescence resonance energy transfer efficiency according to the exosome content according to the present invention;

FIGS. 4A to 4C show graphs illustrating the results of evaluating the cytotoxicity of Examples 1 to 4 and Comparative Examples 1 and 2 according to the present invention in (a) Raw 264.7, (b) HaCaT, and (c) normal human fibroblasts (NHFs);

FIG. 5 shows a graph illustrating the results of evaluating the ability of Examples 1 to 4 and Comparative Examples 1 and 2 according to the present invention to inhibit the production of nitric oxide (NO), an inflammation-related factor;

FIG. 6 shows a graph illustrating the results of evaluating the ability of Examples 1 to 3 and Comparative Example 1 according to the present invention to inhibit the production of tumor necrosis factor-α (TNF-α);

FIG. 7 shows a graph illustrating the results of evaluating the ability of Examples 1 to 3 and Comparative Example 1 according to the present invention to inhibit the production of matrix metalloproteinase (MMP)-1, a skin elasticity-related protein;

FIG. 8 shows a graph illustrating the results of evaluating the ability of Examples 1 to 3 and Comparative Example 1 according to the present invention to produce hyaluronic acid, a type of natural moisturizing factor;

FIG. 9 shows an image illustrating changes in collagen fibers of artificial skin tissue (reconstructed skin tissue) according to the present invention;

FIG. 10A shows an image illustrating the expression of proteinase-activated receptor (PAR)-2, a factor involved in the inflammatory response caused by ultraviolet rays according to the present invention;

FIG. 10B shows a graph illustrating the expression of PAR-2, a factor involved in the inflammatory response caused by ultraviolet rays according to the present invention;

FIG. 11A shows an image illustrating the expression of thymic stromal lymphopoietin (TSLP), a factor involved in the inflammatory response caused by ultraviolet rays according to the present invention;

FIG. 11B shows a graph illustrating the expression of TSLP, a factor involved in the inflammatory response caused by ultraviolet rays according to the present invention;

FIG. 12A shows an image illustrating the expression of interleukin (IL)-6, a factor involved in the inflammatory response caused by ultraviolet rays according to the present invention;

FIG. 12B shows a graph illustrating the expression of IL-6, a factor involved in the inflammatory response caused by ultraviolet rays according to the present invention; and

FIGS. 13A to 13C show graphs illustrating the zeta potential difference distribution of Example 1 and Comparative Examples 1 and 2 according to the present invention.

### DETAILED DESCRIPTION OF EXEMPLARY EMBODIMENTS

Hereinafter, the present invention will be described in detail through examples. The examples described below are merely illustrative of the present invention, and the scope of the present invention is not limited to the examples described below.

### Examples

### Comparative Example 1: Preparation of Hydrangea Exosomes

To separate exosomes from hydrangea leaves, purified water was added to the hydrangea leaves at a weight ratio of 20:1 to soak the hydrangea leaves, and then the hydrangea leaves were ground with a grinder. Thereafter, to remove fibers and debris from the ground hydrangea leaves, the ground hydrangea leaves were centrifuged at 3,000 to 4,000×g for 10 minutes, and only the supernatant excluding the pellet layer was taken to separate and remove the residue. The obtained supernatant was centrifuged using an ultracentrifuge (CP80NX) at 10,000 to 15,000×g for 20 to 30 minutes to obtain a supernatant, and then the supernatant was centrifuged at 100,000 to 150,000×g for two to four hours to increase the purity, and then exosomes settled in a pellet layer. A final exosome-containing pellet layer was resuspended in sterile distilled water and dispersed to prepare exosomes.

### Comparative Example 2: Preparation of Liposomes

Hydrogenated lecithin, terpineol, polyglyceryl-10 stearate, and ceramide N-stearoyl phytosphingosine (NP) were dissolved at 80 to 90° C. or higher, and then purified water, glycerin, 1,2-hexanediol, azelamide monoethanolamine (MEA), and dipotassium glycyrrhizate were heated to 80° C. or higher and then added to the dissolved mixture. After emulsifying at 3,000 rpm for five minutes using a homomixer, the mixture was passed through a high-pressure disperser (microfluidizer) at a pressure of 1,000 bar three successive times, and then cooled and defoamed to obtain a liposome composition, which was prepared to have the composition shown in Table 1 below.

TABLE 1

| Ingredients | Wt % |
|---|---|
| Purified water | Remainder |
| Glycerin | 10.00 |
| 1,2-Hexanediol | 2.00 |
| Hydrogenated lecithin | 1.00 |
| Terpineol | 0.50 |
| Azelamide MEA | 0.50 |
| Polyglyceryl-10 stearate | 0.10 |
| Dipotassium glycyrrhizate | 0.05 |
| Ceramide NP | 0.01 |

### Examples 1 to 4: Preparation of Hybrid Exosomes

To prepare hybrid exosomes, the liposomes and hydrangea exosomes obtained in the above Preparation Examples 1 and 2 were mixed, and the resulting suspension was homogenized by passing it three times at a pressure of 10,000 psi using a microfluidizer, which is a high-pressure homogenizer, to obtain the final hybrid exosomes. Table 2 shows the compositions for preparing the hybrid exosomes according to the present invention.

TABLE 2

| | Liposomes | Hydrangea exosomes |
|---|---|---|
| | (weight ratio) | (weight ratio) |
| Example 1 | 1 | 1 |
| Example 2 | 1 | 4 |
| Example 3 | 1 | 8 |
| Example 4 | 1 | 16 |

### Formulation Example 1: Preparation of Cream

A transparent emulsifying system was prepared by heating and dissolving cetyl ethylhexanoate, cetearyl alcohol, and glyceryl stearate at 75° C. to 80° C. Disodium ethylenediaminetetraacetic acid (EDTA), glycerin, propanediol, cetearyl olivate, sorbitan olivate, and carbomer were dispersed in purified water, and the emulsifying system dissolved in an aqueous phase heated to 70° C. to 75° C. was added, and the resulting mixture was emulsified for five minutes under the conditions of 3,500 to 5,000 rpm with a homomixer. Tromethamine was added at 60° C. to 65° C., and the mixture was stirred for 3 minutes under the conditions of 3,000 to 3,500 rpm with a homomixer to neutralize. 1,2-Hexanediol and ethylhexylglycerin were added at 45° C., and the resulting mixture was stirred for three minutes, and then cooled to 30° C. Thereafter, Example 1 was added, and the resulting mixture was stirred and defoamed for three minutes to prepare a cream formulation. Table 3 below shows the composition for preparing a cream containing the hybrid exosomes of Example 1 according to the present invention.

TABLE 3

| | Formulation Example 1 |
|---|---|
| Ingredients | Content (%) |
| Example 1 | 10 |
| Purified water | Remainer |
| Glycerin | 5 |
| Propanediol | 3 |
| Cetyl ethylhexanoate | 3 |
| Cetearyl alcohol | 2 |
| Cetearyl olivate | 2 |
| Sorbitan olivate | 2 |
| 1,2-Hexanediol | 2 |
| Glyceryl stearate | 0.5 |
| Carbomer | 0.3 |
| Tromethamine | 0.24 |
| Ethylhexylglycerin | 0.05 |
| Disodium EDTA | 0.02 |
| Total | 100 |

### Experimental Example 1: Hybrid Exosome Verification Assay

The verification of hybridization of exosomes and liposomes by the fluorescence resonance energy transfer (FRET) method includes the following three steps: (a) a step of preparing liposomes (fluorescent liposomes) composed of two types of phospholipids conjugated with fluorescent dyes; (b) a step of hybridizing liposomes and the fluorescent liposomes of step (a); and (c) a step of hybridizing liposomes and exosomes. This is illustrated in FIG. 1.

### (a) Preparation of Fluorescent Liposomes (F-Lipo)

Two types of phospholipids conjugated with fluorescent dyes were L-α-phosphatidylethanolamine-N-(4-nitrobenzo-2-oxa-1,3-diazole) (PE-NBD) (ammonium salt) and L-α-phosphatidylethanolamine-N-(lissamine rhodamine B sulfonyl) (PE-Rh-B) (ammonium salt), which are fluorophore lipids of the FRET method and serve as a donor lipid and an acceptor lipid, respectively. 1 ml of a chloroform solution was added to a 50 ml round-bottom flask, and 9 μl of PE-NBD and 90 μl of PE-Rh-B (PE-NBD:PE-Rh-B=1:7 molar ratio) were added and mixed. Using a rotary evaporator, chloroform was slowly evaporated and removed so that the lipid materials in the flask form a thin layer on the inner wall of the flask. 1 ml of distilled water was added to hydrate the lipid, and then extrusion was performed using a syringe extruder (0.4 μm membrane filter) to form fluorescent liposomes.

### (b) Hybridization of Liposomes and Fluorescent Liposomes

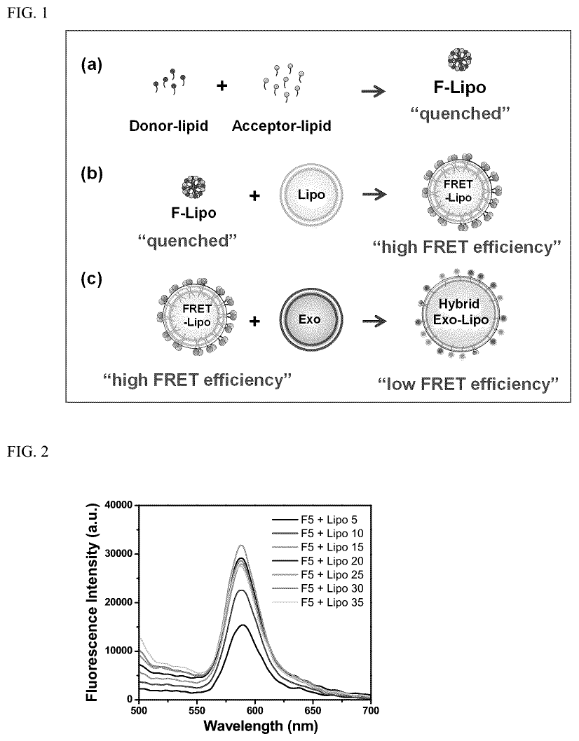

Liposomes (5, 10, 15, 20, 25, 30, 35 μl) were added to a specific amount of fluorescent liposomes (5 μl), and the total volume of the solution was fixed to 1 ml with distilled water, the resulting solution was vortexed for three minutes, and then the fluorescence spectrum of the liposome solution was measured. When the fluorescence spectrum was measured, the excitation/emission wavelengths were fixed at 480 nm/500-700 nm. Since the state change of the solution began after mixing, the measurement was made immediately after mixing, and the measurement time was recorded. As the amount of liposomes increased, the quenched fluorescence signal increased, and the amount of liposomes that exhibited the maximum fluorescence signal intensity was determined. In this example, the maximum fluorescence signal was exhibited in a mixed solution of 5 μl of fluorescent liposomes and 25 μl of liposomes, which is illustrated in FIG. 2.

### (c) Hybridization of Liposomes and Exosomes

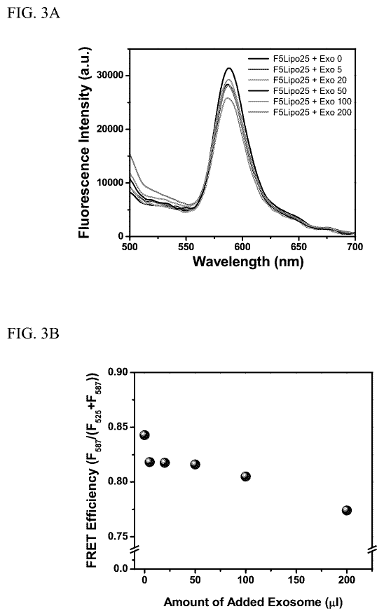

Exosomes of different amounts (0, 5, 20, 50, 100, 200 μl) were added to the fluorescent liposome-liposome solution obtained from step (b) that exhibited the maximum fluorescence signal, and the fluorescence signal was measured. The FRET efficiency below may be calculated from the signal of the fluorescence spectrum. When exosomes are hybridized between fluorescent liposomes, the distance between fluorescent dyes increases, which soon appears as a decrease in FRET efficiency. In other words, a decrease in FRET efficiency indicates an increase in hybridization. As the amount of exosomes added increased, FRET efficiency gradually decreased, confirming that the hybridization of exosomes and liposomes gradually progressed. This is illustrated in FIGS. 3A and 3B and Table 4.

FRET Efficiency=[(F_{A}/(F_{D}+F_{A}))
F_{A}: Fluorescence signal of the acceptor lipid (PE-Rh-B) (at 597 nm)
F_{D}: Fluorescence signal of the donor lipid (PE-NBD) (at 525 nm)

TABLE 4

| Added exosome | FRET efficiency |
|---|---|
| 0 | 0.843 |
| 5 | 0.818 |
| 20 | 0.818 |
| 50 | 0.816 |
| 100 | 0.805 |
| 200 | 0.774 |

### Experimental Example 2: Cytotoxicity Assessment

Normal human fibroblasts (NHFs) were seeded at 6×10³ cells/well, HaCaT cells were seeded at 1.5×10⁴ cells/well, and RAW 264.7 cells were seeded at 1×10⁵ cells/well in 96-well plates, and cultured under cell culture conditions. After 24 hours, the culture medium was discarded, and the cells were washed with phosphate-buffered saline (PBS), and the NHF cells were starved with Fibroblast Growth Medium (FBM) medium containing no supplements, and the HaCaT and RAW264.7 cells were starved with Dulbecco's Modified Eagle Medium (DMEM) containing no fetal bovine serum (FBS). The next day, the cells were treated with the test substances at a certain concentration and cultured for 24 hours. 100 μl of water-soluble tetrazolium (WST)-1 reagent diluted 10-fold in the medium was added to each well, and after two hours of culture, the absorbance was measured at 450 nm.

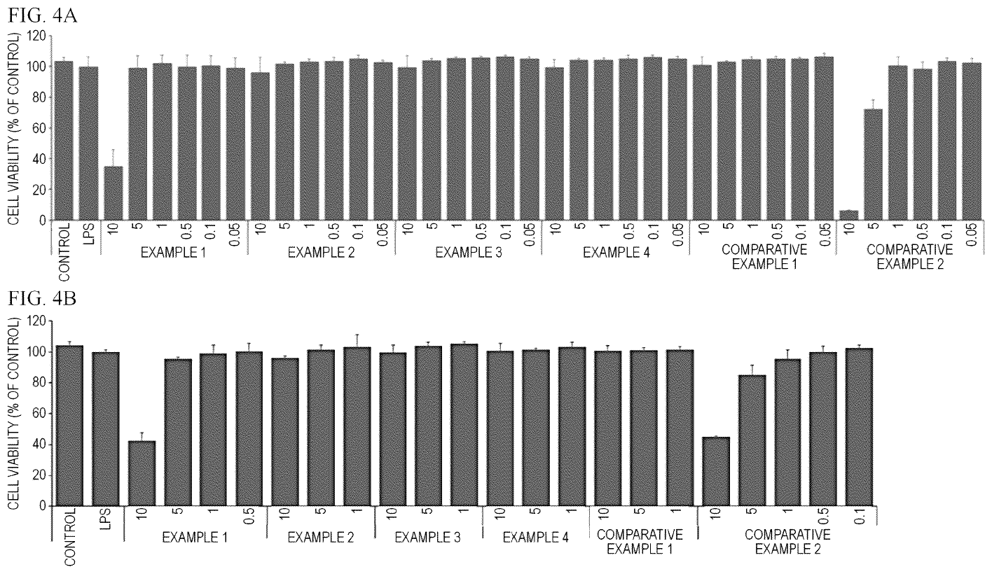

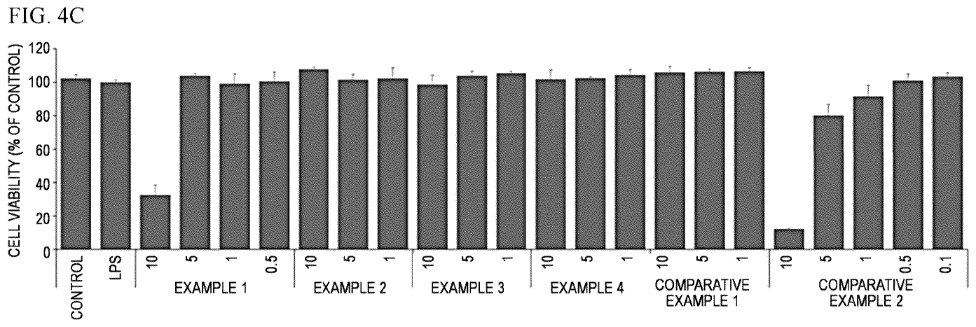

For cytotoxicity tests, the test substances were tested at concentrations ranging from a minimum of 0.05% to a maximum of 10%. As a result, when the NHF, HaCaT, and RAW264.7 cells were each treated with the hybrid exosomes (liposome: hydrangea exosome=1:1, 1:4, 1:8, 1:16), the cell viability was found to be over 90% at the highest concentration of 10%. The concentration of this efficacy test was selected as a cytotoxicity-free concentration to confirm the concentration dependence of the efficacy, which is illustrated in FIGS. 4A to 4C.

### Experimental Example 3: Assessment of Ability to Suppress NO Production

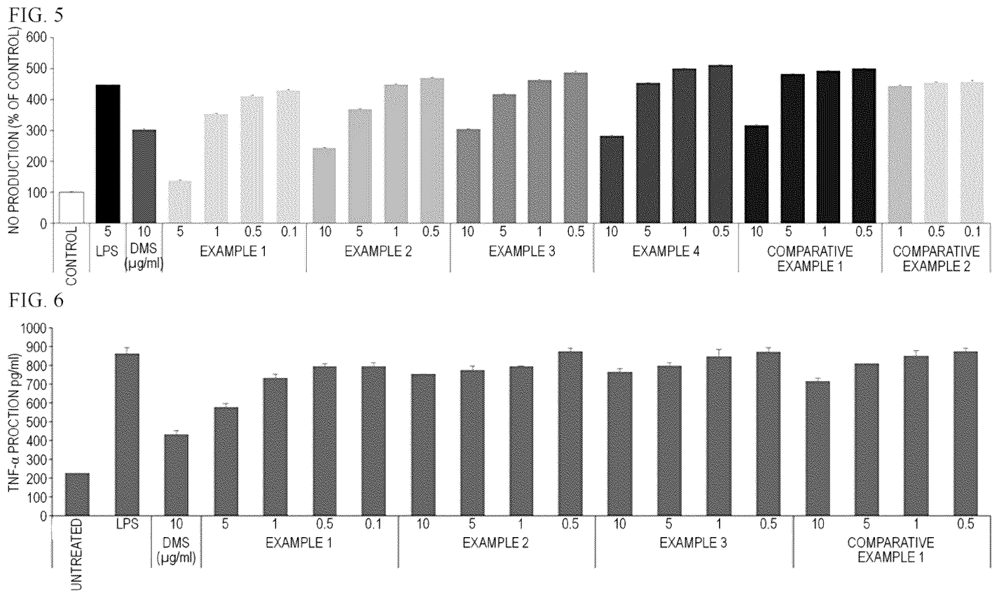

RAW 264.7 cells were seeded at 5×10⁴ cells/well in a 96-well plate and cultured under cell culture conditions. After 24 hours, the culture medium was discarded, and the cells were washed with PBS, and then the cells were starved using DMEM medium containing no FBS. The next day, the cells were treated with 5 μg/ml of lipopolysaccharide (LPS) and cultured with a certain concentration of test substances. After 24 hours, the cell culture medium and the Griess reagent were mixed in equal amounts and allowed to react at room temperature for 15 minutes. The absorbance was measured at 560 nm, and the amount of nitric oxide (NO), an inflammatory mediator, was determined using a standard curve obtained from sodium nitrite. The final amount of NO was converted into the amount of NO per certain amount of protein and compared with the negative control group, and the results are illustrated in FIG. 5.

As a result of the experiment, it was found that the amount of NO produced, which increased 346.98% due to LPS, decreased in a concentration-dependent manner in Examples 1 to 4 and Comparative Examples 1 and 2, and the NO production activity decreased up to 307.92%. Therefore, it was confirmed that the hybrid exosomes have an excellent NO production inhibition effect, and the efficacy according to the optimal mixing ratio could be confirmed.

### Experimental Example 4. Assessment of Ability to Suppress TNF-α Production

The RAW 264.7 macrophage cells were cultured under the conditions of about 37° C. and 5% CO₂ in DMEM supplemented with 10% (v/v) FBS, streptomycin, and penicillin, and seeded in a 96-well plate at an amount of 1×10⁵ cells/well. Then, the cells were treated with each of the compositions prepared according to Examples 1 to 3 and Comparative Example 1 at various concentrations, stimulated with LPS (5 μg/mL), and cultured for 24 hours. The cell culture solution was then collected, and the amount of TNF-α was measured using an enzyme-linked immunosorbent assay (ELISA) kit (R&D Systems Inc. Minneapolis, MN, USA). The amount of TNF-α was quantified using the TNF-α standard curve by concentration included in the kit. Meanwhile, a positive control group was treated with indomethacin (20 μg/mL) instead of the samples and used to compare the TNF-α inhibitory activity with the samples.

As a result of the experiment, the LPS treatment group induced TNF-α production of 865.07 pg/mL, which was confirmed to be a statistically significant increase of about 1.99 times compared to the negative control group (434.01 pg/mL). In Examples 1 to 3, the TNF-α production was 577.41 pg/mL, 753.58 pg/mL, and 766.44 pg/mL. It was found that the TNF-α inhibitory activity of the hybrid exosomes increased in a concentration-dependent manner, and in conclusion, it was confirmed that Example 1 had the best TNF-α inhibitory activity. Therefore, it was confirmed that, compared to using only Comparative Example 1, the use of hybrid exosomes had a better inhibitory effect on TNF-α production, and the efficacy according to the optimal mixing ratio was confirmed, and this is illustrated in FIG. 6.

### Experimental Example 5. Assessment of Ability to Suppress MMP-1 Production

HaCaT cells were seeded at 2.5×10⁵ cells/well in a 96-well plate and cultured under cell culture conditions. After 24 hours, the medium was discarded, and the cells were washed with PBS and starved using DMEM (serum-free medium) containing no FBS. The next day, the cells were cultured while being irradiated with UVB. NHF cells were seeded at 6×10³ cells/well in a 96-well plate and cultured under cell culture conditions. After 24 hours, the cells were starved using an FBM medium that contained no supplements, and the next day, human fibroblasts were treated with the culture medium of HaCaT that had been stimulated by UVB together with the samples and cultured. After 24 hours of culture, the absorbance was measured at 450 nm after an experiment using an MMP-1 ELISA kit. The final amount of MMP-1 was converted into the amount of MMP-1 per certain amount of protein and compared with the negative control group, and the results are illustrated in FIG. 7.

The experimental results showed that the increase in MMP-1 inhibitory activity was significantly greater in Example 1 at the same concentration and under the same conditions. Therefore, it was confirmed that, compared to using only Comparative Example 1, the use of the hybrid exosomes had a better wrinkle improvement effect, and the efficacy according to the optimal mixing ratio was confirmed, and this is illustrated in FIG. 7.

### Experimental Example 6. Assessment of Ability to Produce Hyaluronic Acid

The HaCaT cells were seeded at 1.5×10⁴ cells/well in a 96-well plate and cultured under cell culture conditions. After 24 hours, the culture medium was discarded, and the cells were washed with PBS and starved using DMEM medium containing no FBS. The next day, the cells were treated with the test substances at a certain concentration and cultured for 24 hours. After the experiment using a hyaluronic acid ELISA kit, the absorbance was measured at 450 nm. The final amount of hyaluronic acid was converted into the amount of hyaluronic acid per certain amount of protein and compared with the negative control group.

The experimental results showed that, in the case of Example 1, the amount of hyaluronic acid production increased significantly. Therefore, compared to using only Comparative Example 1, it was confirmed that the use of the hybrid exosomes had a better moisturizing effect, and the efficacy according to the optimal mixing ratio was confirmed, and this is illustrated in FIG. 8.

### Experimental Example 7. Assessment of Efficacy of Alleviating Irritation Caused by Ultraviolet Rays in Artificial Skin

The efficacy of the above-described Preparation Example 1 in alleviating irritation by ultraviolet rays was confirmed using artificial skin. The general morphology of skin tissue and irritation by ultraviolet rays were evaluated by observing the immunostaining of PAR-2, TSLP, and IL-6. First, the artificial skin was treated with ultraviolet rays and samples, and the tissue was recovered 24 hours later. The tissue was fixed in 10% neutral buffered formalin for 24 hours, embedded with the optimal cutting temperature (OCT) compound to prepare 12 μm sections, which were then fixed on slides. After staining using the Fontana-Masson kit and Masson's trichrome stain kit, the morphology was observed under a microscope. After immunofluorescence staining, the morphology was observed under a fluorescence microscope. Statistical analysis was performed using Student's t-test, and a p-value of 0.05 or less was considered statistically significant.

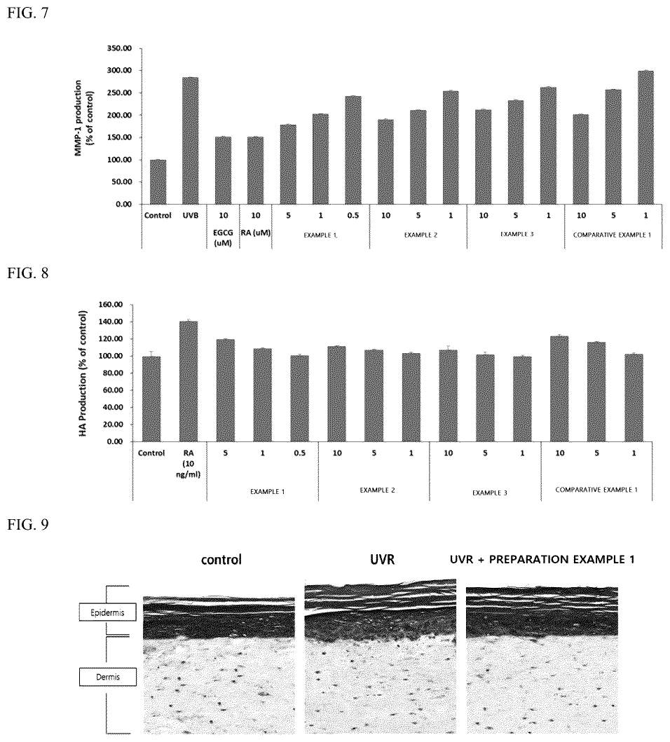

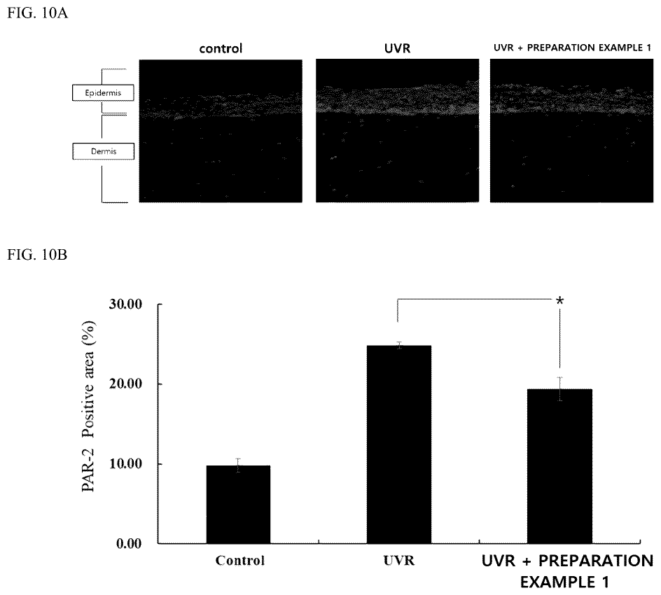

As a result of observing the morphology of the artificial skin, the control group had a thick and slightly laminated stratum corneum, and the epidermis exhibited 5 to 6 cell layers with a good shape. In the dermis, collagen fibers were well observed around fibroblasts, and when treated with ultraviolet rays (UVR), the stratum corneum was damaged and began to spread out, the cells of the epidermis became irregular, and collagen fibers were well observed around fibroblasts in the dermis. In UVR+ Formulation Example 1, the stratum corneum was slightly laminated compared to the group treated with UVR only, and the epidermis exhibited 5 to 6 cell layers with a good shape. It was confirmed that collagen fibers were well observed around fibroblasts in the dermis, and this is illustrated in FIG. 9.

### (1) PAR-2 Assay

When PAR-2 is highly expressed in the skin epidermis, receptor activation becomes prominent in the skin exposed to UV. When the expression and activation of PAR-2 increase, it activates the mechanisms of IκB kinase and nuclear factor KB within the cells, causing an inflammatory response and itching. PAR-2 was stained with red fluorescence in the lower part of the epidermis, and when exposed to UVR stimulation, it was stained with red fluorescence throughout the upper part of the epidermis. After UVR stimulation, it was observed in the sample treatment group that the red fluorescence expression was decreased in the upper part of the epidermis. The blue color indicates the staining of the cell nucleus.

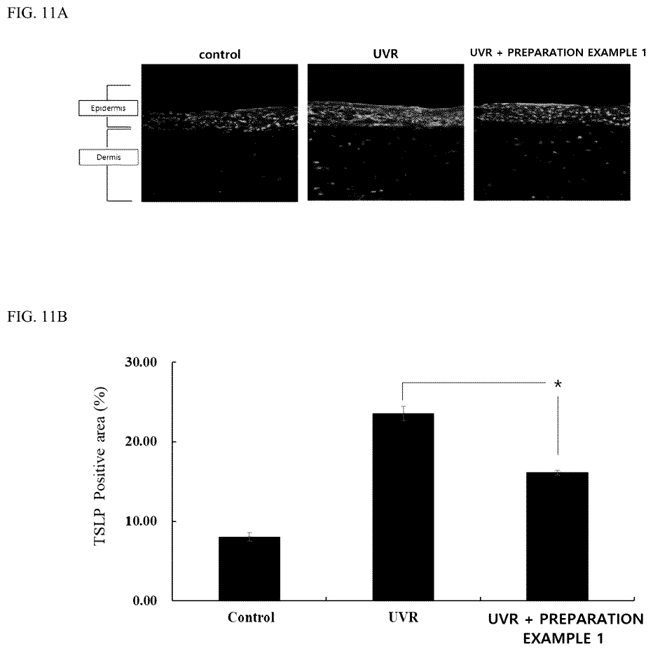

As a result of quantitative analysis, there was a significant change in the PAR-2 positive staining area in the UVR stimulation treatment group compared to the untreated control group, and the increase rate was 153.17%. This indicates that the expression of PAR-2 was increased and activated by UV radiation. Compared to the untreated control group, there was a statistically significant change when Preparation Example 1 was used after the UVR stimulation treatment, and the increase rate was 97.74%. When Preparation Example 1 was used after the UVR stimulation treatment, the expression of PAR-2 was decreased 21.89% compared to the UVR stimulation treatment control group. This indicates that PAR-2 expression by UVR stimulation was inhibited by Preparation Example 1, and this is illustrated in FIG. 10.

### (2) TSLP Assay

TSLP is the first to be activated when the skin is stimulated from the outside, and activates cytokines involved in the inflammatory response. TSLP was stained with green fluorescence throughout the epidermis in the UVR-stimulated control group compared to the untreated control group. In the group treated with the sample after UVR stimulation, it was observed that the green fluorescence expression in the upper part of the epidermis was decreased in the sample-treated group. The blue color indicates the staining of the cell nucleus.

As a result of quantitative analysis, there was a significant change in the TSLP-positive staining area in the UVR stimulation treatment group compared to the untreated control group, and the increase rate was 192.63%. This indicates that TSLP production was increased by UV radiation.

Compared to the untreated control group, there was a statistically significant change when Preparation Example 1 was used after the UVR stimulation treatment, and the increase rate was 100.44%. When Preparation Example 1 was used after the UVR stimulation treatment, the TSLP-positive staining area was decreased 31.50% compared to the UVR stimulation treatment control group. This indicates that the production of TSLP due to UVR stimulation was inhibited by Preparation Example 1, and this is illustrated in FIG. 11.

### (3) IL-6 Assay

IL-6 is a major cytokine of the host response to tissue damage and infection that directly affects the function of skin inflammatory cells. IL-6 was stained with strong green fluorescence throughout the epidermis in the UVR-stimulated control group compared to the untreated control group. IL-6 was also stained with green fluorescence in the cytoplasm of dermal fibroblasts. After UVR stimulation, it was observed that the green fluorescence expression in the epidermis and fibroblast cytoplasm decreased in the sample treatment group. The blue color indicates the staining of the cell nucleus.

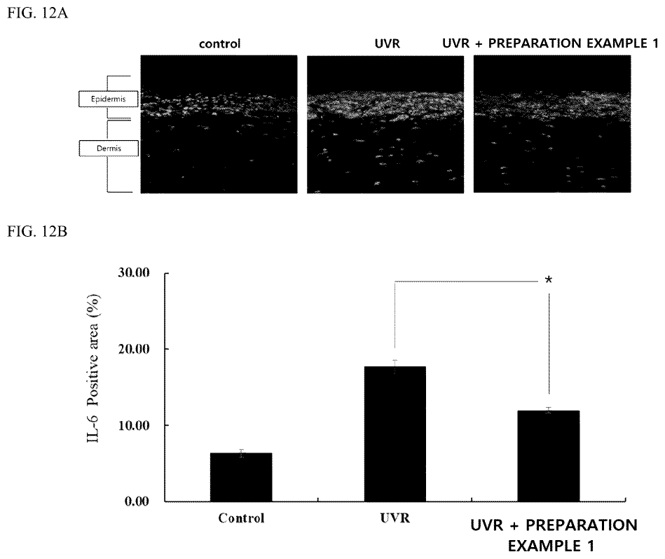

As a result of quantitative analysis, there was a significant change in the IL-6 positive staining area in the UVR stimulation treatment group compared to the untreated control group, and the increase rate was 178.97%. This indicates that the IL-6 production was increased by UV radiation. Compared to the untreated control group, there was a statistically significant change when Preparation Example 1 was used after the UVR stimulation treatment, and the increase rate was 89.05%. When Preparation Example 1 was used after the UVR stimulation treatment, the IL-6 positive staining area was decreased 32.23% compared to the UVR stimulation treatment control group. This indicates that the production of IL-6 due to UVR stimulation was inhibited by Preparation Example 1, and this is illustrated in FIG. 12.

As such, the cosmetic composition including hybrid exosomes of the present invention was confirmed to be effective in anti-inflammation, wrinkle improvement, and moisturization. It was confirmed that the cream formulation containing hybrid exosomes of the present invention exhibited the efficacy of inhibiting the expression of PAR-2, which is a stimulus response factor caused by ultraviolet rays, and the production of TSLP and IL-6 in human-derived skin cell tissue, and thus was effective in alleviating irritation caused by ultraviolet rays.

### Experimental Example 8. Particle Distribution and Electric Potential Measurement

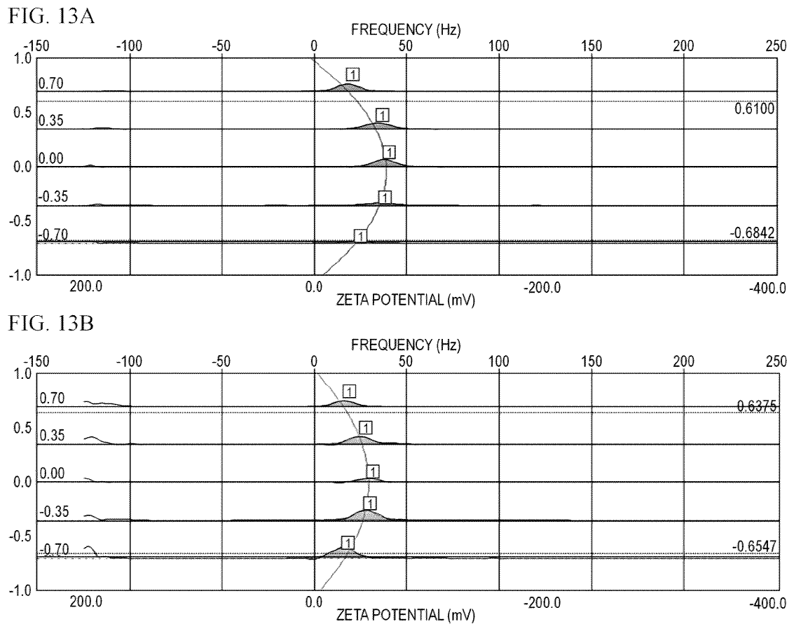

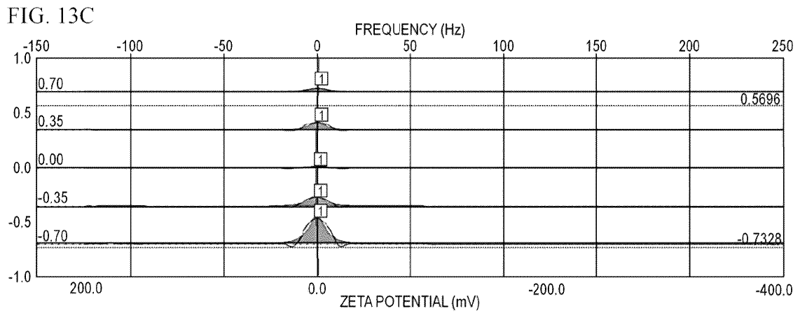

The particle distribution of Example 1, Comparative Example 1, and Comparative Example 2 was measured using Photal. ELS-Z. As a result of the analysis, the particle size was measured to be 169.6 nm, 170.2 nm, and 212.5 nm, respectively, and the results are shown in Table 5. In addition, the electric potential of the particles was −37.44 mV, −0.56 mV, and −28.83 mV, respectively, confirming that Example 1 had the best dispersion stability. The results of Example 1 are illustrated in FIG. 13A, and the results of Comparative Examples 1 and 2 are illustrated in FIGS. 13B and 13C, respectively. In other words, it was confirmed that while the particle size of Comparative Example 1, which was exosomes, was maintained, the stability of Example 1, which was hybrid exosomes, was further improved through membrane fusion.

TABLE 5

| | | Comparative | Comparative |
|---|---|---|---|
| | Example 1 | Example 1 | Example 2 |
| Particle size (nm) | 169.6 | 170.2 | 212.5 |
| Zeta potential (mV) | −37.44 | −0.56 | −28.83 |

The hybrid exosomes provided from the present invention have excellent skin inflammation amelioration, skin wrinkle amelioration, skin moisturization efficacy, and skin regeneration efficacy compared to using hydrangea-derived exosomes and liposomes alone, and thus can be effectively utilized as a cosmetic composition containing the same as an active ingredient.

In addition, the hybrid exosomes are more stable and have superior biocompatibility compared to existing exosomes.

1. Hybrid exosomes in which hydrangea-derived exosomes and liposomes are fused.

2. The hybrid exosomes of claim 1, wherein hydrangea-derived exosomes and liposomes are fused at a weight ratio of 16:1 to 1:2 in the hybrid exosomes.

3. The hybrid exosomes of claim 1, wherein the liposomes include one or more selected from the group consisting of ethanolamide, lecithin, a surfactant, and a ceramide.

4. The hybrid exosomes of claim 3, wherein the ethanolamide is included in an amount of 0.001% to 5% by weight based on the total weight of the hybrid exosomes.

5. The hybrid exosomes of claim 3, wherein the lecithin is included in an amount of 0.1% to 20% by weight based on the total weight of the hybrid exosomes.

6. The hybrid exosomes of claim 3, wherein the surfactant is included in an amount of 0.01% to 20% by weight based on the total weight of the hybrid exosomes.

7. The hybrid exosomes of claim 3, wherein the ceramide is included in an amount of 0.01% to 10% by weight based on the total weight of the hybrid exosomes.

8. The hybrid exosomes of claim 1, wherein the liposomes further include terpineol.

9. The hybrid exosomes of claim 8, wherein the terpineol is included in an amount of 0.01% to 10% by weight based on the total weight of the hybrid exosomes.

10. The hybrid exosomes of claim 1, wherein the diameter of the hybrid exosomes is 50 to 400 nm.

11. A cosmetic composition for improving skin conditions, comprising the hybrid exosomes of claim 1 as an active ingredient.

12. The composition of claim 11, wherein the skin condition improvement is skin inflammation amelioration, skin elasticity improvement, skin wrinkle amelioration, skin regeneration, or skin moisturization.

13. The composition of claim 11, wherein the hybrid exosomes are included in an amount of 0.1% to 10% by weight based on the total weight of the composition.

14. The composition of claim 11, wherein the formulation of the cosmetic composition is any one formulation selected from the group consisting of skin lotion, skin softener, skin toner, astringent, lotion, milk lotion, moisture lotion, nutrition lotion, massage cream, nutrition cream, moisture cream, hand cream, foundation, essence, nutrition essence, pack, soap, cleansing foam, cleansing lotion, cleansing cream, body lotion, body cleanser, facial cleanser, treatment, beauty liquid, beauty pack, ointment, gel, patch, and spray.

15. A composition for delivery of a drug or a bioactive substance, comprising the hybrid exosomes of claim 1; and a drug or a bioactive substance encapsulated within the hybrid exosomes.

16. A method of preparing hybrid exosomes of claim 1, comprising fusing hydrangea-derived exosomes and liposomes.

17. The method of claim 16, wherein the weight ratio of the exosomes and liposomes is 16:1 to 1:2.

18. The method of claim 16, further comprising dispersing terpineol in the liposomes.
